(19) United States
(12) Patent Application Publication
(10) Pub. No.: US 2026/0226115 A1
(43) Pub. Date: Aug. 6, 2026

(54) USE OF MUTANT YAP FOR IMPROVING CARDIAC FUNCTION

(71) Applicants: Baylor College of Medicine, Houston, TX (US); Texas Heart Institute, Houston, TX (US)

(72) Inventors: James F. Martin, Pearland, TX (US); Bing Xie, Houston, TX (US)

(73) Assignees: Baylor College of Medicine, Houston, TX (US); Texas Heart Institute, Houston, TX (US)

(21) Appl. No.: 19/150,445

(22) PCT Filed: Feb. 9, 2024
(86) PCT No.: PCT/US2024/015205

§ 371 Date: Jul. 23, 2025

Related U.S. Application Data
(60) Provisional application No. 63/444,736, filed on Feb. 10, 2023.

(51) Int. Cl. C07K 14/47; A61K 38/17; A61K 48/00; A61P 9/10; C12N 15/86

(52) U.S. Cl. CPC C07K 14/47; A61K 38/1709; A61K 48/0058; A61P 9/10; C12N 15/86; C12N 2750/14143; C12N 2830/008

(57) ABSTRACT

Provided is a modified Yes-associated protein (YAP) in which the serine residues of the LATS1/2 phosphorylation sites and a serine residue in the region of YAP that binds to TEAD are substituted with alanines. Also provided are nucleic acids encoding the modified YAP, vectors comprising the nucleic acids, and compositions comprising the modified YAP, nucleic acids encoding the modified YAP, or vectors comprising the nucleic acids. Further provided are methods of regenerating cardiomyocytes and of treating cardiac conditions.

### CROSS-REFERENCE TO RELATED APPLICATIONS

This application claims priority to U.S. Provisional Application Ser. No. 63/444,736, filed Feb. 10, 2023, which is incorporated by reference herein in its entirety.

### STATEMENT OF GOVERNMENT SUPPORT

This invention was made with government support under grant number R01 HL118761, awarded by the National Institutes of Health. The government has certain rights in the invention.

### BACKGROUND

Myocardial infarction (MI) is a leading cause of death worldwide. During an MI, the contractile myocardium experiences reduced blood flow with decreased oxygen and nutrition supply, eventually causing expansive myocardial cell death. Unlike non-mammalian vertebrates, such as the zebrafish, whose hearts can fully regenerate in responses to injury throughout life, mature mammalian hearts harbor terminally differentiated cardiomyocytes (CMs). CM loss during cardiac ischemia is irreversible and induces cardiac fibroblast activation, which mediates scar formation and leads to impaired cardiac contractility. However, recent studies have demonstrated that the neonatal mouse heart exhibits transient regenerative potential within the first 7 days, suggesting a regulatory mechanism that prevents the adult heart from initiating a regenerative response to ischemia.

The Hippo signaling pathway (HSP), an evolutionarily conserved signaling pathway, was initially identified in Drosophila in its regulation of organ and body size. The core components of HSP in mammals include the sterile 20 (STE20) family protein kinases, encoded by Stk4/3; mammalian STE20-like protein kinase 1/2 (MST1/2), large tumor suppressor kinase 1/2 (LATS1/2), which are phosphorylated and activated by MST1/2; and yes-associated protein (YAP), encoded by Yap1, which is the transcriptional coactivator phosphorylated and inhibited by LATS1/2.

Activated HSP phosphorylates YAP via LATS1/2, and suppresses YAP transcriptional activities by subsequent cytoplasmic retention and degradation. Conversely, in the unphosphorylated state, active YAP interacts with different transcription factions (TFs), such as TEA domain (TEAD) transcription factors, to regulate the expression of diverse genes involved in cell proliferation and cell differentiation.

Previous studies revealed strong HSP activity in postnatal hearts. Deleting the MST1/2 adapter protein Salvador (SAV) in adult mouse hearts causes HSP deficiency and induces a reparative genetic program after MI injury, demonstrating a regulatory role for HSP in cardiac regeneration. Moreover, CM-specific YAP overexpression improves cardiac function and mouse survival by stimulating CM proliferation after MI injury, which further elucidated the mechanism of HSP inhibition on CM renewal.

A CM-specific YAP gain-of-function mouse model, YAP5SA, was previously developed to study YAP activity in greater depth. In the YAP5SA model, the LATS1/2 phosphorylation sites of YAP protein are mutated to completely bypass HSP repression. YAP5SA adult mice exhibit cardiac hyperplasia with thickened ventricular wall and shortened survival. In addition, YAP5SA alters the expression of multiple genes and enhances chromatin accessibility. YAP5SA also initiates both positive and negative feedback loops of CM proliferation. Therefore, an improved YAP mutant that induces CM proliferation without the lethality observed with YAP5SA would be advantageous.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

Described herein is a modified YAP protein, YAP6SA, in which serine residues phosphorylated by LATS1/2 and a serine residue in the region of YAP that binds to TEAD are substituted with alanines. These mutations bypass HSP inhibition regulation and inhibit the interaction between YAP and TEAD. In some embodiments, YAP6SA promotes CM proliferation and, unlike YAP5SA, its expression is well-tolerated in mammalian hearts. In addition, YAP6SA improves cardiac repair post-MI injury with decreased scar area and improved heart contractility.

Accordingly, in one embodiment, provided is a YAP variant comprising the amino acid sequence set forth in SEQ ID NO: 3, a nucleic acid encoding the YAP variant, and/or a vector comprising the nucleic acid. In certain embodiments, the vector is a viral vector. In a particular embodiment, the vector is an AAV vector, such as an AAV9 vector.

In some embodiments, the nucleic acid is operably linked to a cell- or tissue-specific promoter, for example, a cardiac-specific promoter. In one embodiment, the promoter is a cardiomyocyte-specific promoter.

Also provided are compositions comprising the YAP variant, the nucleic acid encoding the YAP variant, or the vector comprising the nucleic acid, along with a carrier. In a preferred embodiment, the composition is a pharmaceutical composition.

Methods of using the YAP variant are also provided. One aspect is a method of regenerating cardiomyocytes in a subject in need thereof, the method comprising delivering to cardiac tissue of the subject a composition of the invention. Another aspect is a method of treating MI in a subject, the method comprising delivering to cardiac tissue of the subject a composition of the invention. The composition can be administered to the subject more than once.

Specific embodiments of the invention include a method of regenerating cardiomyocytes in a subject and/or a method of treating MI in a subject, the method comprising delivering to cardiac tissue of the subject a pharmaceutical composition comprising an AAV vector, wherein the vector comprises a nucleic acid encoding SEQ ID NO: 3 under the control of a cardiomyocyte-specific promoter.

Examples of subjects for whom methods of the invention would be beneficial include subjects having arrhythmia, cardiomyopathy, heart failure, myocardial fibrosis, or myocarditis, and/or subjects who have experienced MI.

It is contemplated that any aspect discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

FIGS. 1A-1J show analysis of YAP6SA-overexpressing mouse phenotype. FIG. 1A shows AAV9-GFP, AAV9-YAP5SA, and AAV9-YAP6SA expression cassettes; cTnT: cardiac troponin T. FIG. 1B shows immunofluorescence staining of Flag expression in mice overexpressing YAP5SA (YAP5SA OE) or YAP6SA (YAP6SA OE) at 1-4 days post-AAV9 infection and of cTnT expression at 4 days post-infection. FIG. 1C shows Western blotting results of Flag-YAP5SA and Flag-YAP6SA expression levels in postnatal murine hearts. FIG. 1D shows co-immunoprecipitation results of YAP5SA and YAP6SA interactors in murine hearts. FIG. 1E shows immunofluorescence staining of Flag expression in YAP5SA OE and YAP6SA OE mice at 3 days post-AAV9 infection. FIG. 1F shows quantification of Flag signal location in YAP5SA OE and YAP6SA OE CMs (n=6 each). FIG. 1G shows a survival curve of GFP, YAP5SA OE, and YAP6SA OE mice (n=8 each). FIG. 1H shows echocardiography results in AAV9-GFP and AAV9-YAP6SA infected mice at 4 and 8 weeks post-administration: EF: ejection fraction, FS: fractional shortening. FIG. 1I shows H&E histology showing the cardiac structure of GFP and YAP6SA OE mice at 4 weeks post-AAV9 infection. FIG. 1J shows H&E histology of cross-sections of GFP and YAP6SA OE hearts at 4 weeks post-infection.

FIGS. 2A-2D show that YAP6SA promotes cardiomyocyte proliferation. FIG. 2A shows immunofluorescence staining for PCNA and quantification of PCNA-positive cardiomyocytes (n=6 each). FIG. 2B shows immunofluorescence staining for pHH3 and quantification of pHH3-positive cardiomyocytes (n=6 each). FIG. 2C shows immunofluorescence staining for Aurora B and quantification of Aurora B-positive cardiomyocytes (n=6 each). **: p<0.01, ***: p<0.001. FIG. 2D shows immunofluorescence staining for Confetti mouse heart sections and quantification of total clones (n=6 each).

FIGS. 3A-3E show that YAP6SA regulates expression of multiple genes in cardiomyocytes. FIG. 3A shows a bulk RNA-seq experiment pipeline. FIG. 3B shows a heatmap of differential gene expression in AAV9-GFP CMs, AAV9-YAP5SA OE CMS, and AAV9-YAP6SA OE CMs. FIG. 3C shows a split violin plot showing average fold change for each gene cluster in YAP5SA OE CMs and YAP6SA OE CMs. FIG. 3D shows gene ontology analysis of differentially expressed genes (DEGs) in YAP5SA OE CMs and YAP6SA OE CMs. FIG. 3E shows predicted upstream regulators of DEGs in AAV9-YAP6SA-overexpressing CMs.

FIGS. 4A-4H show that YAP6SA interacts with diverse protein factors in cardiomyocytes. FIG. 4A shows a plot of YAP6SA interactome in CMs. FIG. 4B shows the intersection of established YAP, YAP5SA, and YAP6SA interactomes. FIG. 4C shows classification of YAP6SA interactors in CMs. FIG. 4D shows gene ontology analysis of DEGs in YAP6SA OE CMs. FIG. 4E shows interaction of YAP6SA with MPDZ in CMs by immunoprecipitation. FIG. 4F shows quantification of Rhos signal intensity in dividing CMs in YAP6SA OE hearts. FIG. 4G shows experimental design of ROCK inhibitor treatment and quantification of immunofluorescence staining results comparing pHH3 positive CMs among groups. (n=6 each). FIG. 4H shows a model of YAP6SA's role in CM proliferation.

FIGS. 5A-5J show that YAP6SA stimulates cardiac regenerative responses post-injury. FIG. 5A shows a first timeline for MI surgery, AAV9 infection, and sample analysis. FIG. 5B shows trichrome histology of AAV9-GFP and AAV9-YAP6SA mice at 4 weeks post-MI injury, and FIG. 5C shows fibrosis quantification. FIG. 5D shows echo wave images of GFP and YAP6SA MI mice. Echo-cardiography results in FIG. 5E show cardiac function in MI-GFP and MI-YAP6SA at 4 weeks post-injury. FIG. 5F shows cardiac remodeling in YAP6SA MI hearts. FIG. 5G shows a second timeline for MI surgery, AAV9 infection, and sample analysis. FIG. 5H shows trichrome histology of AAV9-GFP and AAV9-YAP6SA mice at 3 weeks post-MI injury, and FIG. 5I shows fibrosis quantification. FIG. 5J shows echo wave images and cardiac function in GFP and YAP6SA MI mice at 3 weeks post-injury.

FIGS. 6A-6E show that YAP6SA stimulates cardiac regenerative responses in adult mice. FIG. 6A shows a timeline for MI surgery, AAV9 infection, and analysis. FIG. 6B shows trichrome histology of AAV9-GFP and AAV9-YAP6SA mice at 3 weeks post-MI injury, and FIG. 6C shows fibrosis quantification. FIG. 6D shows echo wave images of GFP and YAP6SA MI mice. Echo-cardiography results in FIG. 6E show cardiac function in MI-GFP and MI-YAP6SA at 3 weeks post-injury.

### DETAILED DESCRIPTION OF THE INVENTION

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods and compositions of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the compositions and processes of the present invention are their ability to induce CM proliferation or regeneration or otherwise stimulate cardiac regenerative response(s) to improve cardiac function for treatment and/or prevention of a cardiac condition.

Wherever embodiments are described with the language “comprising,” otherwise analogous embodiments described in terms of “consisting of” and/or “consisting essentially of” are included. Thus, in any of the claims, the term “consisting of” or “consisting essentially of” can be substituted for any of the open-ended linking verbs recited above, in order to change the scope of a given claim from what it would otherwise be using the open-ended linking verb. Additionally, the term “wherein” may be used interchangeably with “where”.

Units, prefixes, and symbols are denoted in their Système International d'Unités (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

The terms “about” or “approximately” are defined as being close to as understood by one of ordinary skill in the art. In one non-limiting embodiment the terms are defined to be within 10%, within 5%, within 1%, or within 0.5%.

Human YAP1 has the amino acid sequence set forth in SEQ ID NO: 1. The LATS phosphorylation sites are underlined. Serine residues at positions 61, 109, 127, 128, 131, 163, 164, and 381, shown in bolded italics, are substituted with alanine in YAP5SA.

Compared to YAP5SA, YAP6SA contains an additional serine-to-alanine substitution at position 94, in the YAP/TEAD interface. The ability of YAP6SA to function in the absence of its interaction with TEAD was unexpected, based on literature reports of this being an obligate interaction for YAP function in the heart (Zhao et al. 2008; Li et al. 2010; von Gise et al. 2012). As demonstrated in the Examples, YAP6SA induces CM proliferation and improves cardiac function, and, unlike YAP5SA, is safe and well-tolerated.

| (SEQ ID NO: 1) | |
|---|---|
| MDPGQQPPPQ PAPQGQGQPP SQPPQGQGPP SGPGQPAPAA TQAAPQAPPA | 50 |
| GHQIVHVRGD SETDLEALFN AVMNPKTANV PQTVPMRLRK LPD [symbol] FFKPPE | 100 |
| PKSHSRQAST DAGTAGALTP QHVRAHSSPA SLQLGAVSPG TLTPTGVVSG | 150 |
| PAATPTAQHL RQSSFEIPDD VPLPAGWEMA KISSGQRYFL NHIDQTTTWQ | 200 |
| DPRKAMLSQM NVTAPISPPV QQNMMNSASG PLPDGWEQAM TQDGEIYYIN | 250 |
| HKNKTTSWLD PRLDPRFAMN QRISQSAPVK QPPPLAPQSP QGGVMGGSNS | 300 |
| NQQQQMRLQQ LQMEKERLRL KQQELLRQEL ALRSQLPTLE QDGGTQNPVS | 350 |
| SPGMSQELRT MIINSSDPFL NSGTYHSRDE STDSGLSMSS YSVPRTPDDF | 400 |
| LNSVDEMDTG DTINQSTLPS QQNRFPDYLE AIPGINVDLG ILEGDGMNIE | 450 |
| GEELMPSLQE ALSSDILNDM ESVLAATKLD KESFLIWL | 488 |

Accordingly, human YAP5SA has the amino acid sequence shown in SEQ ID NO: 2, and human YAP6SA has the amino acid sequence shown in SEQ ID NO: 3. Substitutions in the wild-type sequence are shown in bolded italics.

| (SEQ ID NO: 2) | |
|---|---|
| MDPGQQPPPQ PAPQGQGQPP SQPPQGQGPP SGPGQPAPAA TQAAPQAPPA | 50 |
| GHQIVHVRGD AETDLEALFN AVMNPKTANV PQTVPMRLRK LPDSFFKPPE | 100 |
| PKSHSRQAAT DAGTAGALTP QHVRAHAAPA ALQLGAVSPG TLTPTGVVSG | 150 |
| PAATPTAQHL RQAAFEIPDD VPLPAGWEMA KTSSGQRYFL NHIDQTTTWQ | 200 |
| DPRKAMLSQM NVTAPISPPV QQNMMNSASG PLPDGWEQAM TQDGEIYYIN | 250 |
| HKNKTTSWLD PRLDPRFAMN QRISQSAPVK QPPPLAPQSP QGGVMGGSNS | 300 |
| NQQQQMRLQQ LQMEKERLRL KQQELLRQEL ALRSQLPTLE QDGGTQNPVS | 350 |
| SPGMSQELRT MITNSSDPFL NSGTYHSRDE ATDSGLSMSS YSVPRTPDDF | 400 |
| LNSVDEMDTG DTINQSTLPS QQNRFPDYLE AIPGTNVDLG TLEGDGMNIE | 450 |
| GEELMPSLQE ALSSDILNDM ESVLAATKLD KESFLTWL | 488 |
| (SEQ ID NO: 3) | |
| MDPGQQPPPQ PAPQGQGQPP SQPPQGQGPP SGPGQPAPAA TQAAPQAPPA | 50 |
| GHQIVHVRGD AETDLEALFN AVMNPKTANV PQTVPMRLRK LPDAFFKPPE | 100 |
| PKSHSRQAAT DAGTAGALTP QHVRAHAAPA ALQLGAVSPG TLTPTGVVSG | 150 |
| PAATPTAQHL RQAAFEIPDD VPLPAGWEMA KTSSGQRYFL NHIDQTTTWQ | 200 |
| DPRKAMLSQM NVTAPTSPPV QQNMMNSASG PLPDGWEQAM TQDGEIYYIN | 250 |
| HKNKTTSWLD PRLDPRFAMN QRISQSAPVK QPPPLAPQSP QGGVMGGSNS | 300 |
| NQQQQMRLQQ LQMEKERLRL KQQELLRQEL ALRSQLPTLE QDGGTQNPVS | 350 |
| SPGMSQELRT MTTNSSDPFL NSGTYHSRDE ATDSGLSMSS YSVPRTPDDF | 400 |
| LNSVDEMDTG DTINQSTLPS QQNRFPDYLE AIPGTNVDLG TLEGDGMNIE | 450 |
| GEELMPSLQE ALSSDILNDM ESVLAATKLD KESFLTWL | 488 |

Corresponding murine YAP5SA and YAP6SA amino acid sequences are set forth in SEQ ID NO: 4 and SEQ ID NO: 5, respectively. Substitutions in the wild-type sequence are shown in bolded italics.

| (SEQ ID NO: 4) | |
|---|---|
| DPGQQPPPQP APQGQGQPPS QPPQGQGPPS GPGQPAPAAT QAAPQAPPAG | 50 |
| HQIVHVRGDA ETDLEALFNA VMNPKTANVP QTVPMRLRKL PDSFFKPPEP | 100 |
| KSHSRQAATD AGTAGALTPQ HVRAHAAPAA LQLGAVSPGT LTPTGVVSGP | 150 |
| AATPTAQHLR QAAFEIPDDV PLPAGWEMAK TSSGQRYFLN HIDQTTTWQD | 200 |
| PRKAMLSQMN VTAPTSPPVQ QNMMNSASGP LPDGWEQAMT QDGEIYYINH | 250 |
| KNKTTSWLDP RLDPRFAMNQ RISQSAPVKQ PPPLAPQSPQ GGVMGGSNSN | 300 |
| QQQQMRLQQL QMEKERLRLK QQELLRQELA LRSQLPTLEQ DGGTQNPVSS | 350 |
| PGMSQELRTM TTNSSDPFLN SGTYHSRDEA TDSGLSMSSY SVPRTPDDFL | 400 |
| NSVDEMDTGD TINQSTLPSQ QNRFPDYLEA IPGTNVDLGI LEGDGMNIEG | 450 |
| EELMPSLQEA LSSDILNDME SVLAATKLDK ESFLTWL | 487 |
| (SEQ ID NO: 5) | |
| DPGQQPPPQP APQGQGQPPS QPPQGQGPPS GPGQPAPAAT QAAPQAPPAG | 50 |
| HQIVHVRGDA ETDLEALFNA VMNPKTANVP QTVPMRLRKL PDAFFKPPEP | 100 |
| KSHSRQAATD AGTAGALTPQ HVRAHAAPAA LQLGAVSPGT LTPTGVVSGP | 150 |
| AATPTAQHLR QAAFEIPDDV PLPAGWEMAK TSSGQRYFLN HIDQTTTWQD | 200 |
| PRKAMLSQMN VTAPISPPVQ QNMMNSASGP LPDGWEQAMT QDGEIYYINH | 250 |
| KNKITSWLDP RLDPRFAMNQ RISQSAPVKQ PPPLAPQSPQ GGVMGGSNSN | 300 |
| QQQQMRLQQL QMEKERLRLK QQELLRQELA LRSQLPTLEQ DGGTQNPVSS | 350 |
| PGMSQELRTM TTNSSDPFLN SGTYHSRDEA TDSGLSMSSY SVPRTPDDFL | 400 |
| NSVDEMDTGD TINQSILPSQ QNRFPDYLEA IPGTNVDLGT LEGDGMNIEG | 450 |
| EELMPSLQEA LSSDILNDME SVLAATKLDK ESFLTWL | 487 |

Provided herein are YAP variants comprising the amino acid sequence set forth in SEQ ID NO: 3 or in SEQ ID NO: 5, along with nucleic acids encoding these sequences. As used herein, the term “nucleic acid” refers to a polymer of DNA or RNA having a combination of purine and pyrimidine bases, sugars, and covalent linkages between nucleosides including a phosphate group in a phosphodiester linkage. A nucleic acid can be single-stranded or double-stranded, and will optionally contain synthetic, non-natural, or altered nucleotide bases capable of incorporation into DNA or RNA polymers.

The nucleic acid can be operably linked to an expression control sequence, such that the coding sequence is under the transcriptional control of the expression control sequence. The expression control sequence is preferably a promoter, for example, a tissue-specific promoter. Promoters specific for expression in cardiac tissue or cardiomyocytes include cardiac troponin T promoters, such as the chicken cardiac troponin T (cTnT) promoter, myosin light chain 2 (MLC-2v) promoter; alpha myosin heavy chain (MHC) promoter; and the minimum promoter from −137 to +85 of NCX1 promoter.

Also provided are vectors comprising a nucleic acid encoding a YAP variant of the invention, preferably under transcriptional control of a promoter. Suitable vectors include, for example, viral vectors, non-viral vectors, and non-integrating vectors. Viral vectors can be adenoviral vectors, adeno-associated viral (AAV) vectors, or retroviral vectors, such as a lentiviral vector. In specific embodiments, the vector is an AAV vector. The AAV vector can be of any serotype, including, for example, AAV2, AAV6, AAV7, AAV8, and AAV9. In one embodiment, an AAV9 vector is employed. Non-viral vectors include plasmid, cosmid, phage, bacterial, yeast, and cell vectors.

In certain embodiments, the YAP variant, the nucleic acid encoding the YAP variant, or the vector comprising the nucleic acid encoding the YAP variant, are comprised in a composition, such as a pharmaceutical composition. In some aspects, the YAP variant, the nucleic acid encoding the YAP variant, the vector comprising the nucleic acid encoding the YAP variant, or a composition of any of the foregoing, is administered to a subject (e.g., a mammalian subject, such as a human subject). In some aspects, a nucleic acid encoding the YAP variant is translated in vivo to produce the YAP variant. The nucleic acid encoding the YAP variant may be induced for translation of the YAP variant in a cell, tissue, or organism. In exemplary embodiments, such translation occurs in vivo, e.g., in cardiac tissue (e.g., myocardium) or cardiac cells (e.g., CMs), although there can be envisioned embodiments where such translation occurs ex vivo, in culture or in vitro. In exemplary embodiments, the cell, tissue, or organism is contacted with an effective amount of the YAP variant, the nucleic acid encoding the YAP variant, or the vector comprising the nucleic acid encoding the YAP variant.

The term “pharmaceutical composition” refers to a preparation that is in such form as to permit the biological activity of the active ingredient to be effective, and which contains no additional components that produce an adverse, allergic, or other untoward reaction or are unacceptably toxic to a subject to which the composition would be administered. Pharmaceutical compositions typically comprise a pharmaceutically acceptable carrier and can comprise one or more of a buffer (e.g., acetate, phosphate or citrate buffer), a surfactant (e.g., polysorbate), a stabilizing agent (e.g., human albumin), a preservative (e.g., benzyl alcohol), and/or other conventional solubilizing or dispersing agents. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in the compositions disclosed herein is contemplated. Supplementary active ingredients, such as other treatments for cardiac conditions, can also be incorporated into the compositions. Choice of dosage form and excipients depends upon the active agent to be delivered and the disease or disorder to be treated or prevented and is routine to one of ordinary skill in the art.

Pharmaceutical compositions disclosed herein are suitable for parenteral administration. Parenteral routes of administration include intravenous, intramuscular, intraperitoneal, intrathecal, and subcutaneous. In a preferred embodiment, the pharmaceutical composition is suitable for local administration to the cardiac tissue of a patient. “Local administration” means that a pharmaceutical composition is administered directly to where its action is desired (e.g., at or near the site of the injury or symptoms). One route of administration is direct injection of the pharmaceutical composition into cardiac tissue, for example, the myocardium. It is within the purview of one of ordinary skill in the art to formulate pharmaceutical compositions that are suitable for their intended route of administration. Pharmaceutical compositions of the invention can be administered to a patient once or more than once.

An “effective amount” of a composition as disclosed herein is an amount sufficient to carry out a specifically stated purpose. An “effective amount” can be determined empirically, in relation to the stated purpose, route of administration, and dosage form. In some embodiments, an “effective amount” is an amount sufficient to ameliorate at least one symptom, behavior, or event, associated with a pathological, abnormal, or otherwise undesirable condition, or an amount sufficient to prevent or lessen the probability that such a condition will occur or re-occur, or an amount sufficient to delay worsening of such a condition. For instance, in some embodiments, the effective amount refers to the amount of the YAP variant, the nucleic acid encoding the YAP variant, or the vector comprising the nucleic acid encoding the YAP variant, or composition of any of the foregoing that can induce CM proliferation or regeneration or otherwise stimulate cardiac regenerative responses to improve cardiac function for treatment and/or prevention of a cardiac condition in a subject.

By “subject” or “individual” or “patient” is meant a mammalian subject for whom diagnosis, prognosis, or therapy is desired. In a preferred embodiment, the mammalian subject is a human.

The YAP variants of the invention, nucleic acids encoding the YAP variants, vectors comprising nucleic acids encoding the YAP variants, and compositions of the invention can be used in a method of regenerating cardiomyocytes in a subject; in a method of treating arrhythmia, cardiomyopathy, heart failure, myocardial fibrosis, myocardial infarction, or myocarditis in a subject; in a method of manufacturing a medicament for regenerating cardiomyocytes in a subject; and/or in a method of manufacturing a medicament for treating arrhythmia, cardiomyopathy, heart failure, myocardial fibrosis, myocardial infarction, or myocarditis in a subject.

Accordingly, one embodiment is a method of regenerating cardiomyocytes or treating a cardiac condition in a subject in need thereof, the method comprising administering to heart tissue of the subject a composition comprising YAP6SA, a nucleic acid encoding YAP6SA, or a vector comprising a nucleic acid encoding YAP6SA. A further embodiment is a method of treating arrhythmia, cardiomyopathy, heart failure, myocardial fibrosis, myocardial infarction, or myocarditis in a subject in need thereof, the method comprising administering to heart tissue of the subject a composition comprising YAP6SA, a nucleic acid encoding YAP6SA, or a vector comprising a nucleic acid encoding YAP6SA. In some embodiments, the composition is administered to CMs in the subject.

In some embodiments, a subject in need of the compositions or methods of the invention has a cardiac condition that would be improved by the compositions and methods of the invention, for example, wherein the cardiac condition can be improved by regeneration of cardiomyocytes in the subject. In some embodiments, the cardiac condition comprises arrhythmia; heart failure; cardiomyopathy, such as age-related cardiomyopathy, diabetic cardiomyopathy, dilated cardiomyopathy, ischemic cardiomyopathy; myocardial fibrosis; myocardial necrosis; myocarditis; or myocardial infarction. In some embodiments, the subject has arrhythmia; heart failure; cardiomyopathy, such as age-related cardiomyopathy, diabetic cardiomyopathy, dilated cardiomyopathy, ischemic cardiomyopathy; myocardial fibrosis; myocardial necrosis; or myocarditis. In some embodiments, the subject has experienced MI.

Terms such as “inhibiting” or “reducing” or “preventing” or “avoiding” or any variation thereof, when used in the claims and/or the specification, indicate an approach for preventing, inhibiting, or reducing the likelihood of the occurrence or recurrence of, a disease or condition, e.g., a cardiac condition, and include any measurable decrease in the intensity, effect, symptoms, and/or burden of a disease or condition (e.g., at least, at most, exactly, or between any two of a 1%, 5%, 10%, 15%, 20%, 25%, 30%, 35%, 40%, 45%, 50%, 55%, 60%, 65%, 70%, 75%, 80%, 85%, 90%, 95%, or 99% decrease) or complete (i.e., 100%) inhibition thereof. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, delaying the rate of disease progression, amelioration or palliation of the disease state, and remission or improved prognosis. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder. Accordingly, “treatment” does not necessarily indicate complete eradication or cure of the disease or condition, or associated symptoms thereof.

In the present context, successful treatment can include, for example, improved heart function, such as improved systolic function, improved capillary formation in heart tissue, and/or reduction of arrhythmia. Improvement in heart function in an individual subjected to treatment with a composition or method of the invention is relative to heart function in the individual prior to treatment with a composition or method of the invention. Heart function can be assessed, for example, by measuring ejection fraction, fractional shortening levels, diastolic volume, systolic volume, left ventricular end-diastolic volume, left ventricular end-systolic volume, left ventricular systolic function, diastolic function, stroke volume, cardiac rhythm, or combinations thereof.

Reduction of arrhythmia in a patient is assessed as frequency and/or number of arrhythmias in a patient subjected to treatment with a composition or method of the invention, relative to frequency and/or number of arrhythmias in the patient prior to treatment with a composition or method of the invention.

Subjects treated by compositions and methods of the invention can experience reduced fibrosis of cardiac tissue. Fibrosis can be assessed by measuring heart function and/or visually, for example, by computer tomography, echocardiology, endomyocardial biopsy, and/or magnetic resonance imaging. Reduced fibrosis in an individual subjected to treatment with a composition or method of the invention is relative to fibrosis in the individual prior to treatment with a composition or method of the invention.

Embodiments of the present disclosure can be further defined by reference to the following non-limiting examples. The examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, it will be apparent to those skilled in the art that many modifications, both to materials and methods, can be practiced and still obtain a like or similar result without departing from the scope of the present disclosure. Efforts have been made to ensure accuracy with respect to numbers used (e.g., amounts, temperatures, etc.), but some experimental error and deviation should, of course, be allowed for.

### EXAMPLES

### Example 1. Generation of a YAP6SA Gain-of-Function Expression Cassette

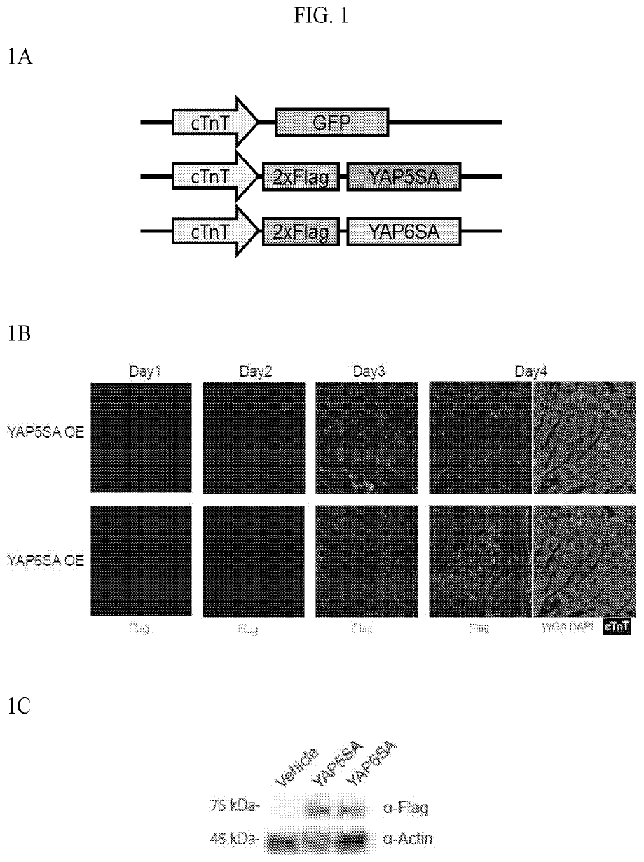

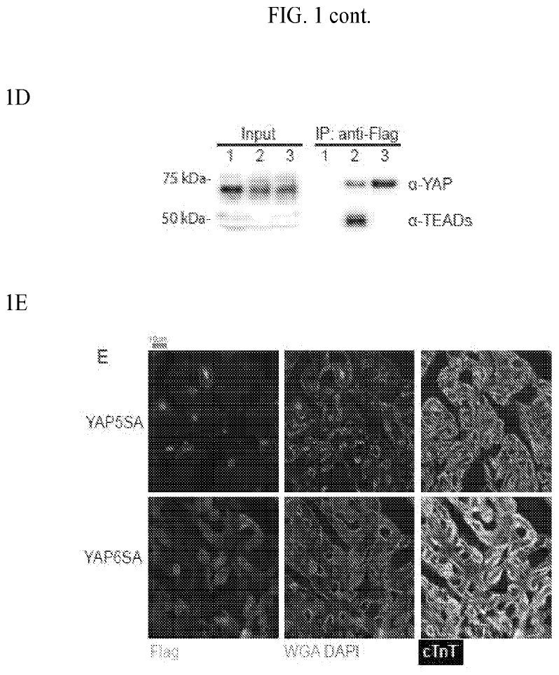

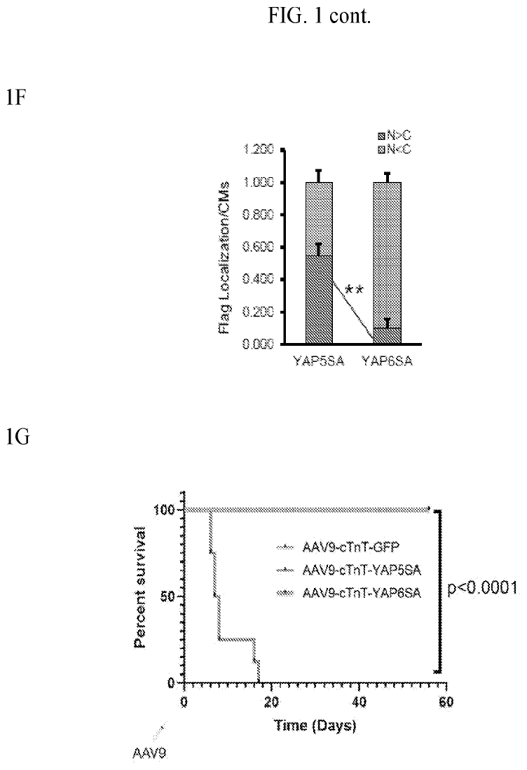

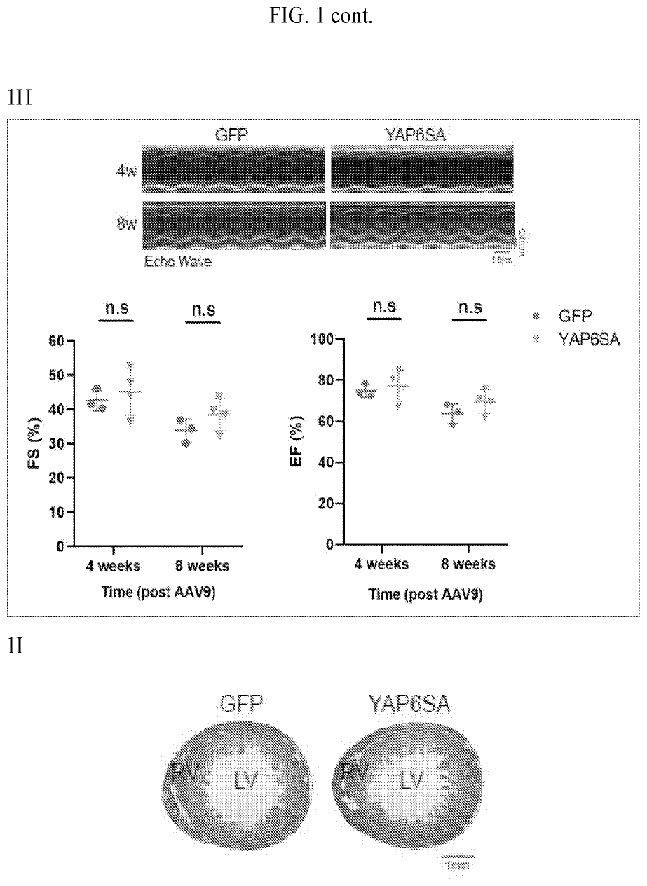

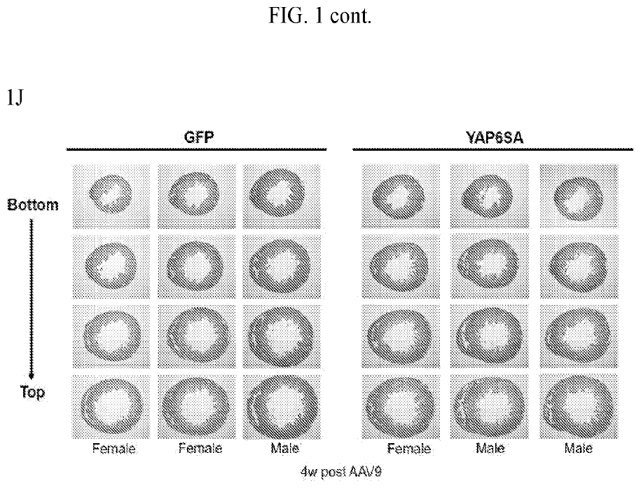

A YAP6SA expression cassette in which YAP6SA is tagged with Flag protein and transcribed by the cardiomyocyte-specific cardiac troponin T promoter (cTnT) was generated (FIG. 1A). YAP6SA harbors nine point mutations, eight of which are the same as in YAP5SA. In both YAP5SA and YAP6SA, the LATS1/2 phosphorylation sites are mutated from serine to alanine. YAP6SA has an additional serine-to-alanine mutation in the region of the YAP/TEAD interface.

YAP5SA and YAP6SA expression cassettes were packaged in adeno-associated virus serotype 9 (AAV9) vectors, and 6-day-old mice (P6) were infected with the AAV9 vectors. To confirm YAP5SA and YAP6SA expression in cardiomyocytes (CMs), immunofluorescence (IF) was performed to detect Flag protein signal at different time points post-AAV9 infection, from day 1 to day 7. Cardiac troponin T (cTnT) was used as the CM marker.

YAP5SA and YAP6SA were expressed widely at day 3 (FIG. 1B). Blotting results demonstrated that YAP5SA and YAP6SA exhibited similar expression levels in postnatal murine hearts after AAV9 delivery (FIG. 1C). Co-immunoprecipitation confirmed the disrupted interaction between TEAD and YAP6SA (FIG. 1D). While Flag-YAP5SA and Flag-YAP6SA were both expressed specifically in CMs, YAP6SA exhibited a decreased localization ratio in the nucleus and an increased ratio in the cytoplasm (FIG. 1E, 1F). These results suggest that YAP/TEAD interaction regulates YAP nuclear translocation.

### Example 2. YAP6SA Overexpression is Safely Tolerated In Vivo

To determine the effect of YAP6SA overexpression (OE) on heart development, mouse survival was examined among AAV9-GFP, AAV9-YAP5SA, and AAV9-YAP6SA OE groups. All YAP6SA OE mice and control (GFP) mice survived for at least 60 days post-administration, indicating that YAP6SA was well tolerated in vivo. In contrast, YAP5SA was lethal in all YAP5SA OE mice by 17 days post-administration. Survival curves are shown in FIG. 1G.

To determine whether YAP6SA impacts murine cardiac function, echocardiography was performed to measure the ejection fraction (EF) and fractional shortening (FS) levels of GFP and YAP6SA OE hearts 4 weeks and 8 weeks after AAV9 delivery. YAP6SA OE mice had similar FS levels (about 40%) and EF levels (about 75%) as AAV9-GFP control mice and similar echocardiograms (FIG. 1H). Furthermore, histology sections of heart demonstrated that YAP6SA did not alter cardiac structure 4 weeks post-AAV9 infection (FIG. 1I, 1J). Together, these data indicate that YAP6SA OE is well tolerated in vivo and does not impair cardiac structure and function in mammals.

### Example 3. Cardiac-Specific YAP6SA Promotes Cardiomyocyte Proliferation

To investigate whether YAP6SA promotes CM division, immunofluorescence (IF) analysis of mouse hearts was performed at 3 days post-AAV9 infection to examine CM cell cycle status using three cell-cycle markers: proliferating cell nuclear antigen (PCNA), phosphorylated Histone H3-Ser10 (pHH3), and Aurora B. PCNA plays an essential role in DNA synthesis and is highly expressed in S phase. In M phase, pHH3 is expressed during chromosome condensation (prophase) and persists throughout metaphase before declining at anaphase. The chromosomal passenger protein Aurora B is detected more broadly throughout cytokinesis.

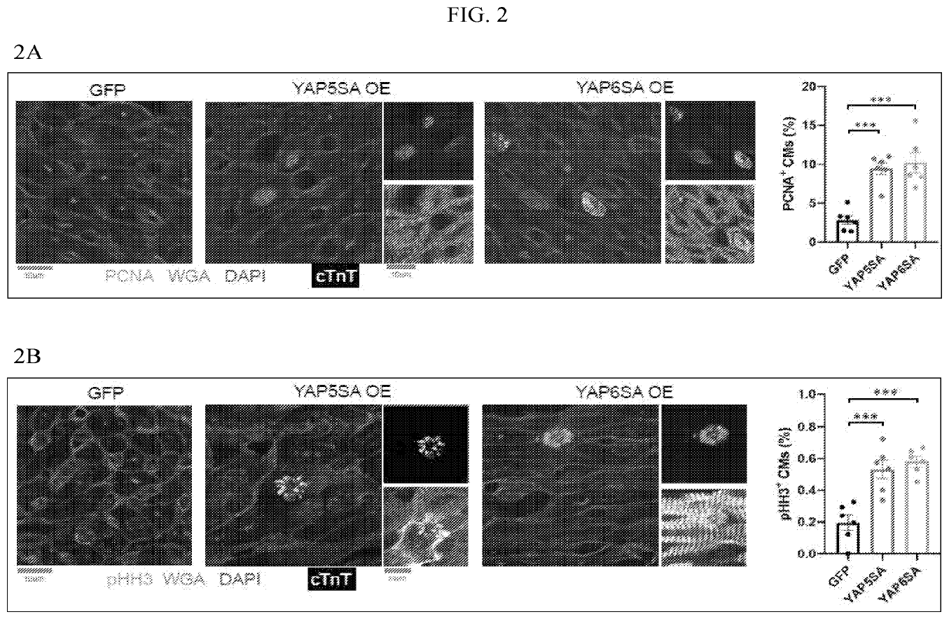

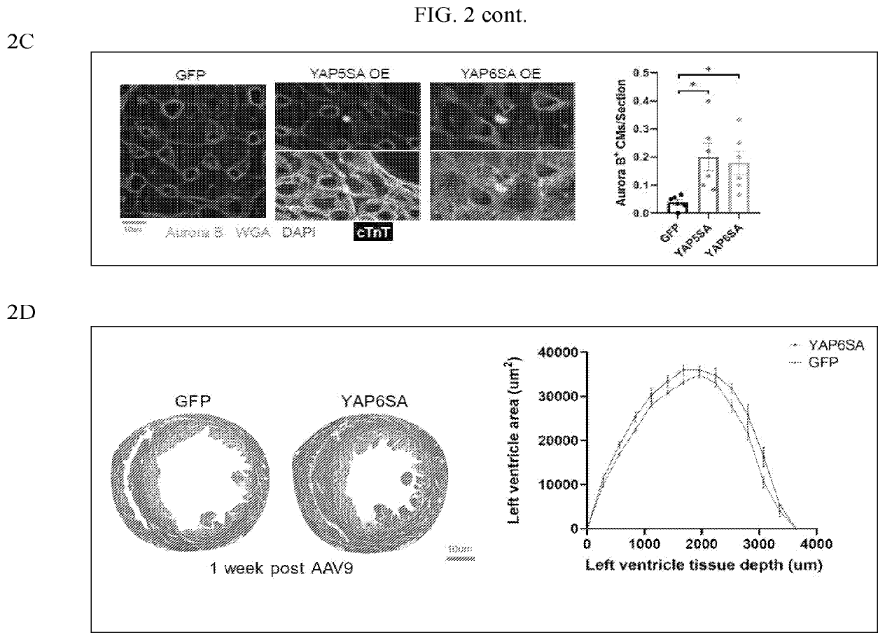

IF results showed increased CM proliferation in YAP6SA OE hearts compared to the AAV9-GFP control, with approximately 10% PCNA-positive CMs (FIG. 2A), 0.6% pHH3-positive CMs (FIG. 2B), and 0.2 Aurora B-positive CMs (FIG. 2C) per area in the hearts. In addition, YAP6SA OE hearts had similar numbers of dividing CMs compared to YAP5SA OE hearts.

To further confirm enhanced CM proliferation, YAP6SA OE hearts were collected after a week of AAV9 infection and analyzed via histology to compare the thickness of cardiac ventricular walls between GFP control and YAP6SA OE hearts. YAP6SA OE hearts have thickened ventricular walls (FIG. 2D). These results show that YAP6SA promotes CM cell cycle progression.

### Example 4. YAP6SA Alters Diverse Gene Expression in Cardiomyocytes

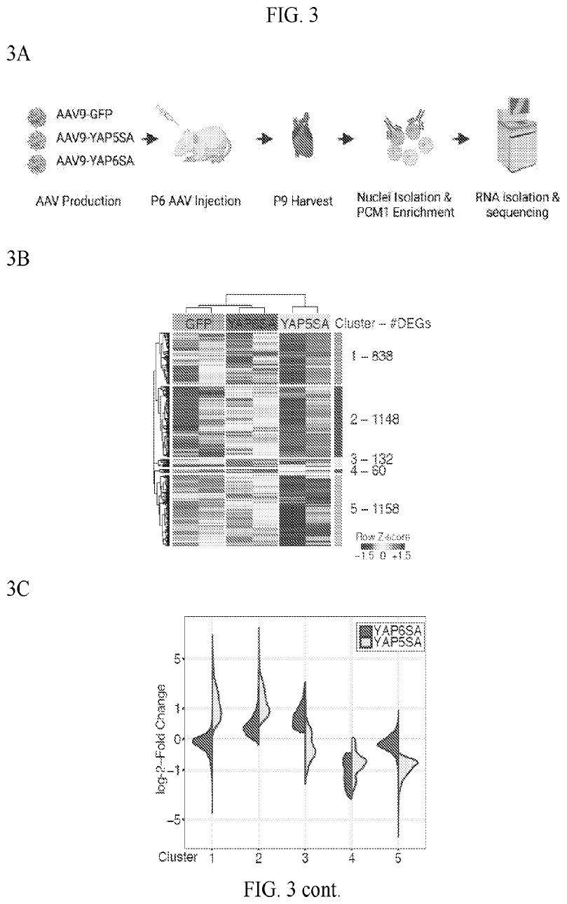

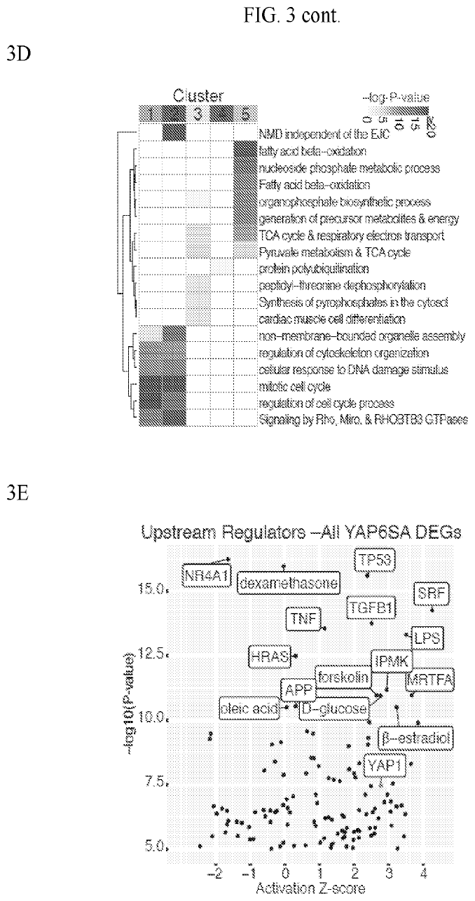

To investigate the mechanism of YAP6SA function, gene expression in YAP6SA OE CMs was compared to that in GFP control and YAP5SA OE CMs. CM nuclei were harvested 3 days post-AAV9 infection, and RNA was extracted for transcriptional profiling (FIG. 3A). The results in YAP6SA OE CMs show five distinct clusters of differentially expressing genes (DEGs) among GFP, YAP5SA and YAP6SA OE CMs. YAP6SA OE CMs have less DEGs compared to YAP5SA OE CMs, including 209 genes that were up-regulated and 96 genes that were down-regulated (FDR<0.05) (FIG. 3B, 3C). This data indicates decreased transcriptional activity in YAP6SA OE CMs versus YAP5SA OE CMs.

In addition, Gene Ontology (GO) analysis revealed that YAP5SA-specific upregulated genes are mainly involved in cell cycle progression, and that the downregulated genes are related to oxidative metabolism, while YAP6SA-specific upregulated genes participate in TCA cycle and CM differentiation. Notably, YAP5SA and YAP6SA OE CMs shared some DEGs that are relevant to cytoskeletal organization. The GO analysis for YAP6SA OE versus GFP CMs confirmed the increased expression of actin and microtubule-related genes, such as Acta1, Actg1, Myl9 and Rhoa. Additionally, some upregulated genes, including Myh8 and Srf, mediate muscle cell differentiation and development; and some genes, such as Pdk4 participate in the tricarboxylic acid (TCA) metabolic cycle, (FIG. 3D). Moreover, YAP6SA-repressed genes, such as Csf1 and Arel1, are involved in the immune response.

The predicted upstream regulators of DEGs in YAP6SA OE CMs include YAP (FIG. 3E), which confirmed the RNA-seq results. These data suggest a TEAD-independent role of YAP6SA in reorganizing CM cytoskeleton structure.

### Example 5. YAP6SA Interacts with Multiple Protein Factors in Cardiomyocytes

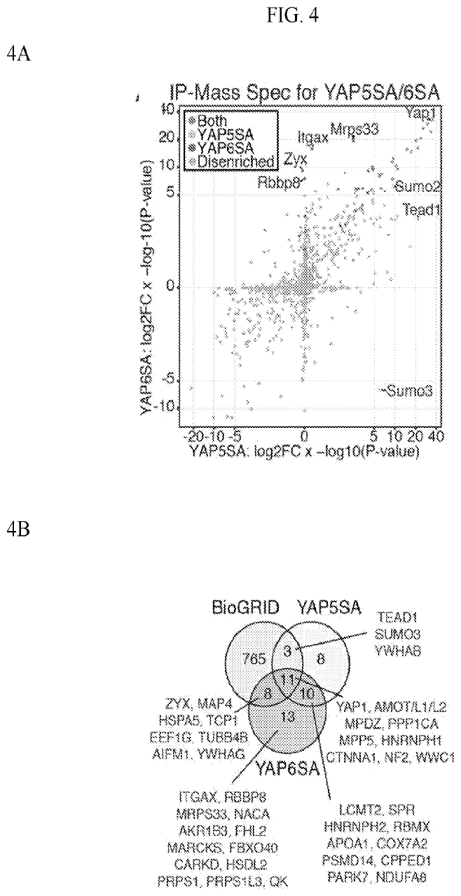

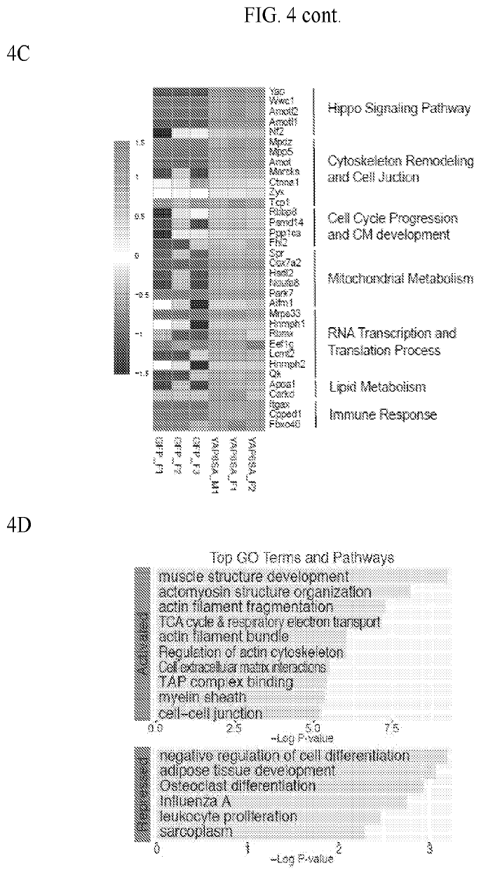

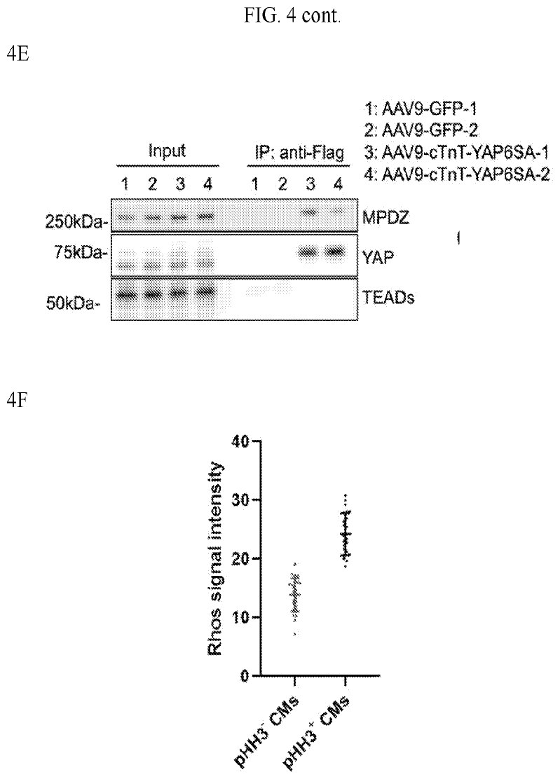

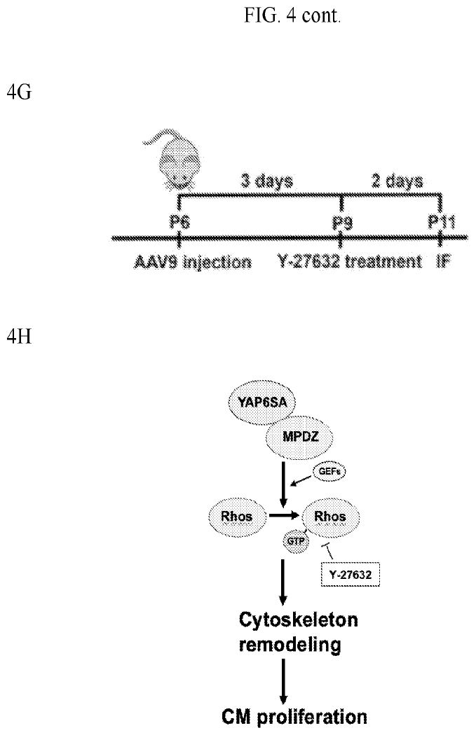

YAP6SA interactors in CMs were further identified by applying an anti-Flag antibody to pull down Flag-tagged YAP6SA and its interacting proteins from murine hearts at 3 days post-AAV9 infection for mass-spectrometry analysis. Results showed that YAP6SA interacted with diverse protein factors (FIG. 4A, 4B). For example, YAP6SA bound to classical Hippo pathway components WW domain-containing protein/kidney and brain expressed protein (WWC1/KIBRA), angiomotin-like protein 2 (AMOTL2), and neurofibromatosis type-2 protein (NF2), which regulate LATS1/2 and YAP/transcriptional coactivator with PDZ-binding motif protein (TAZ) activities.

YAP6SA also cooperated with retinoblastoma-binding protein 8 (RBBP8), proteasome 26S non-ATPase subunit 14 (PSMD14), protein phosphatase 1 catalytic subunit alpha (PPP1CA), and the transcription co-factor four and a half LIM domains 2 (FHL2), all of which are related to cell cycle progression and CM development. Interestingly, YAP6SA formed complexes with mitochondrial ribosomal protein S33 (MRPS33), heterogeneous nuclear ribonucleoprotein H1 (HNRNPH1), RNA binding motif protein X-linked (RBMX), eukaryotic elongation factor-1 gamma (EEF1G), phosphoribosyl pyrophosphate synthetase 1 (PRPS1), and phosphoribosyl pyrophosphate synthetase 1-like 3 (PRPS1L3), all of which are involved in RNA transcription and translation, suggesting an alternative function of YAP6SA. Proteins participating in oxidative metabolism in mitochondria, cytochrome C oxidase subunit 7A2 (COX7A2), NADH-ubiquinone oxidoreductase subunit A8 (NDUFA8), and Parkinson's disease protein 7 (PARK7), were also YAP6SA interactors. Moreover, multiple PDZ domain protein (MPDZ), membrane protein palmitoylated 5 (MPP5), myristoylated alanine rich c-kinase substrate (MARCKS), αE-catenin (CTNNA1), and zyxin (ZYX), which have been shown to modulate cytoskeleton and cell junction organization, were significant YAP6SA partners (FIG. 4C).

The combination of mass-spec and RNA-seq results indicate that YAP6SA has multiple TEAD-independent functions in regulating various CM activities, and according to the increased ratio of YAP6SA localization in cytoplasm, YAPSA appears to have a major role in regulating CM cytoskeleton structure and in providing an appropriate environment for CM cell cycle progression.

### Example 6. YAP6SA Promotes Cardiomyocyte Proliferation by Activating Rho GTPases

The mass spectroscopy results demonstrated that the MPDZ protein has a strong binding affinity for YAP6SA. MPDZ, also named MUPP1, contains 13 PDZ domains and serves as a scaffolding protein to help organize higher-order protein complexes and maintain cell polarity. MPDZ has also been shown to coordinate with Rho guanine nucleotide exchange factors (GEFs) to activate Rho GTPases in the endothelial cell migration and in the vicinity of synapses. Rho family proteins control almost all fundamental cellular processes in eukaryotes, and regulate cytokinesis in certain cell types.

To determine if YAP6SA stimulates CM cell cycle re-entry by increasing Rho GTPases activity, co-immunoprecipitation of YAP6SA-MPDZ in CMs (FIG. 4E) and expression levels of Rho family genes among GFP, YAP5SA OE, and YAP6SA OE groups were analyzed. Indeed, the Rho genes, especially Rhobtb1 and Rhoa, were upregulated in YAP6SA OE groups. Dividing CMs in YAP6SA OE hearts had increased Rho proteins activity (FIG. 4F).

To explore whether inhibiting Rho GTPases activity disrupts YAP6SA functions, the ROCK inhibitor, Y-27632, was delivered to YAP6SA OE mouse hearts, the number of pHH3 positive CMs was measured two days after (FIG. 4G). Indeed, pHH3-positive CMs were significantly decreased in YAP6SA OE hearts treated with Y-27632, compared to the YAP6SA OE hearts with treated with DMSO. The data indicate that YAP6SA enhances CM proliferation by activating Rho-GTPases (FIG. 4H).

### Example 7. YAP6SA OE Hearts have Improved Regeneration Capacity Post-MI

Fully developed mammalian CMs lose the ability of self-renewal, which leads to scar formation and impaired cardiac function after MI injury. Since YAP6SA promoted re-entry of CMs into the cell cycle, it was investigated whether YAP6SA could provoke cardiac regenerative responses to ischemic injury in murine hearts.

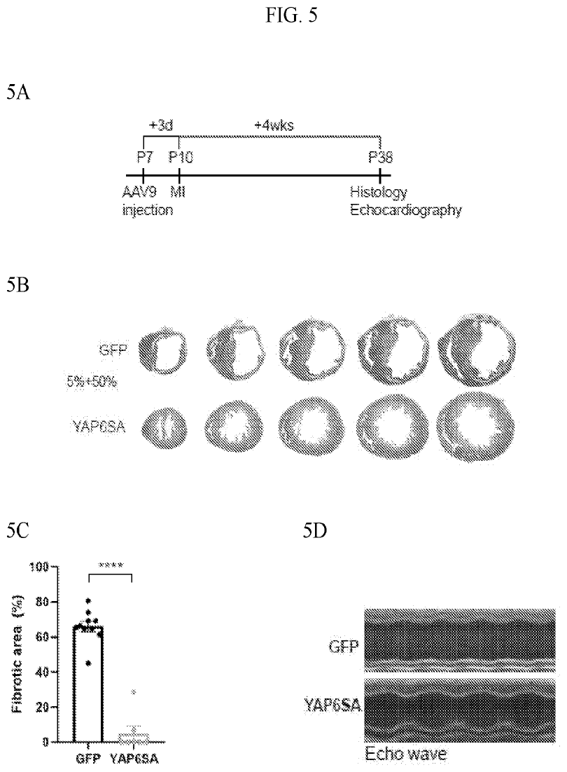

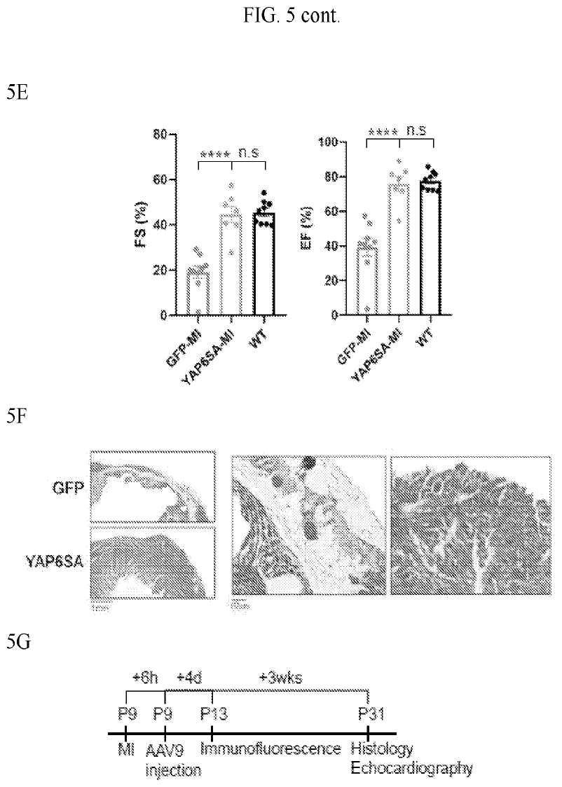

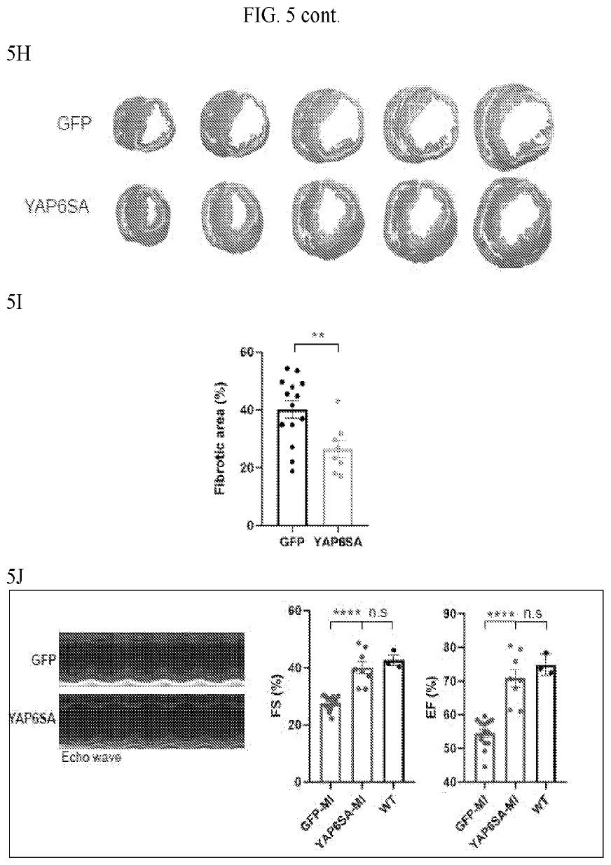

Seven-day-old (P7) mice were infected with AAV9-GFP or AAV9-YAP6SA and underwent surgically induced MI by permanent occlusion of the left anterior descending coronary artery at 3 days post-AAV9 infection. Hearts were analyzed at 4 weeks post-surgery (FIG. 5A). YAP6SA MI mice exhibited significantly decreased scar area compared to control MI mice (FIG. 5B, 5C). YAP6SA OE hearts also displayed enhanced cardiac ejection fraction and fractional shortening levels post-MI, suggesting that YAP6SA improved cardiac function post-injury (FIG. 5D, 5E). Additionally, cardiac remodeling was reduced in YAP6SA MI hearts, demonstrating YAP6SA-induced CM regrowth when under ischemia stress (FIG. 5F).

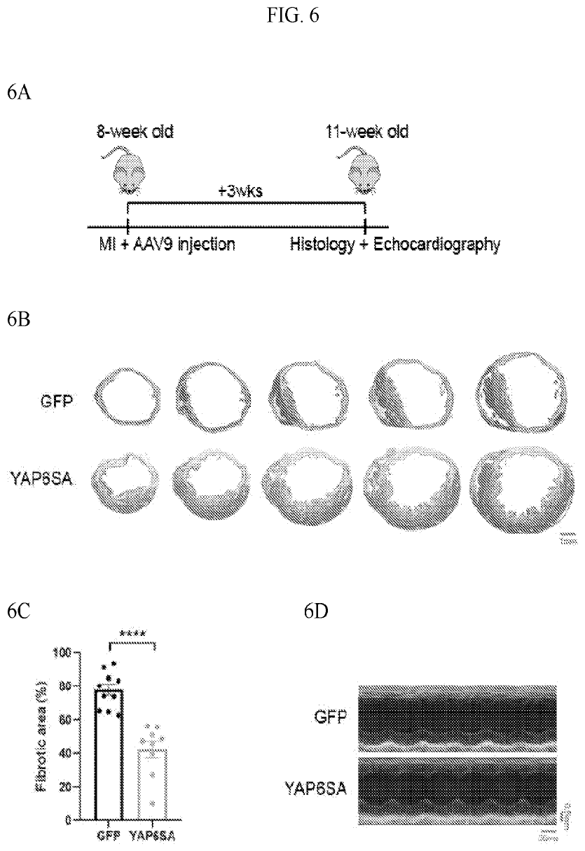

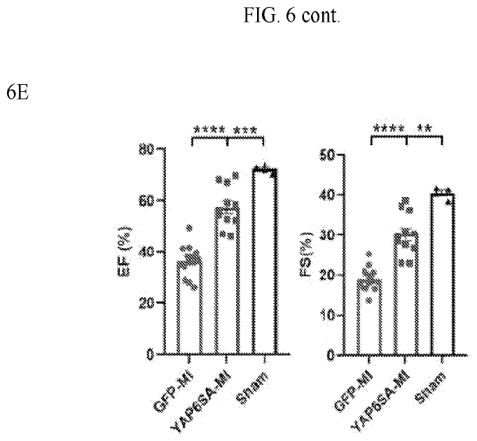

In another experiment, MI injury was induced 6 hours before AAV9 virus infection, and MI hearts were analyzed at 3 weeks post-injury (FIG. 5G). The results were consistent, demonstrating that YAP6SA delivery after MI reduced cardiac fibrosis and rescued heart contractility compared to the GFP control group (FIG. 5H-5J). Furthermore, MI surgery was performed on 8-week-old mice, which were then infected with AAV9-GFP or AAV9-YAP6SA. YAP6SA enhanced cardiac repair in adult mouse hearts at four weeks post-infection (FIG. 6A-6E).

These findings support the conclusion that YAP6SA improves mammalian cardiac repair post-ischemic injury.

### Example 8. Materials and Methods

### Animals

Mice were housed and maintained in accordance with recommendations set in the Guide for the Care and Use of Laboratory Animals of the National Institutes of Health. All animal protocols were approved by the Baylor College of Medicine Institutional Animal Care and Use Committee (IACUC). Male and female mice were used for all experiments except for adult mouse MI surgery, in which all mice were males. Mice were maintained on FVB or ICR background. All control animals were littermates or age-matched if littermates were unavailable.

### Surgical Procedures

To induce myocardial infarction in 8 to 10-week-old mice, the left anterior descending artery was permanently ligated as previously described. Briefly, mice were anaesthetized with 2% isoflurane and then intubated. The heart was exposed by performing a thoracotomy through the fourth or fifth intercostal space. An 8-0 nylon suture was tied around the left anterior descending artery. The clinical definition of heart failure is a 20% reduction in left ventricular ejection fraction as indicated on echocardiography (that is, from >50% ejection fraction to <40% ejection fraction in humans).

To induce myocardial infarction in P8 postnatal mouse hearts, the left anterior descending artery was permanently ligated as previously described. Briefly, mice were anaesthetized with hypothermia, the heart was exposed via thoracotomy through the fourth or fifth intercostal space, and an 8-0 nylon suture was tied around the left anterior descending coronary artery.

### AAV

Constructs containing the GFP, YAP5SA, or YAP6SA gene sequence were cloned into the pENN.AAV.cTNT vector, which is transcribed under the cTnT promoter. All vectors were packaged into the muscle-trophic serotype AAV9 by the Intellectual and Developmental Disabilities Research Center Neuroconnectivity Core at the Baylor College of Medicine. After titering, viruses were aliquoted, immediately frozen, and placed at −80° C. for long-term storage. For subcutaneous injection into postnatal mice, each aliquot was diluted in saline to a 50 μl volume. A total of 1×10¹¹ viral genomes was delivered to each mouse. For retro-orbital injection into the adult MI mouse model, each aliquot was diluted in saline to a 100 μl volume. A total of 3×10¹¹ viral genomes was delivered to each mouse.

### Co-Immunoprecipitation

Mouse whole hearts were homogenized and lysed using RIPA lysis buffer [10 mM Tris-Cl at pH 8.0, 140 mM NaCl, 1 mM EDTA, 1% Triton X-100, 0.1% sodium deoxycholate, 0.1% SDS, 1× protease inhibitor cocktail, and 1× phosphatase inhibitor (Roche)]. Lysates were centrifuged for 20 min. at 12000 rpm, and supernatants were collected for immunoprecipitation. YAP5SA and YAP6SA and their interacting proteins were purified using Anti-FLAG M2 Magnetic Beads (Sigma) for 4-hour rotated incubation at 4° C. The beads were washed three times, for 10 min. each, using RIPA lysis buffer and boiled with elution buffer (4×loading:RIPA=1:3) for 10 min. The antibodies used for immunoblotting in this context were rabbit anti-YAP (1:2000), Novus Biologicals Cat #NB110-583538; rabbit anti-TEADs (D3F7L) (1:1000), Cell Signaling Technology Cat #13295; and rabbit anti-MUPP1/MPDZ (1:1000), Invitrogen Cat #42-2700.

### Western Blotting

Western blotting was performed using standard methods with lysates prepared by homogenizing hearts with a homogenizer in RIPA buffer. After boiling for 5 min. in a reducing tris-based sodium dodecyl sulfate (SDS) sample buffer, the lysates were loaded into acrylamide gels and run at 120 volts for a sufficient time to achieve separation. Proteins were then transferred to PVDF membranes and imaged using the Amersham imager 680 system (GE Healthcare). Primary antibodies were as follows: rabbit anti-YAP (1:1000), Novus Biologicals Cat #NB110-583538; rabbit anti-DYKDDDDK Tag (D6W5B) (1:2000), Cell Signaling Technology Cat #14973; Rabbit anti-GAPDH (1:3000), Abcam; mouse anti-β-actin (C4) (1:3000), Santa Cruz Biotechnology Cat #sc-47778. HRP-conjugated secondary antibodies were goat anti-rabbit IgG (H+L) and goat anti-mouse IgG (H+L) (1:5000), Jackson ImmunoResearch Cat #111-035-003. Quantitation was performed using the gel analysis feature in Fiji (ImageJ) (National Institutes of Health, Bethesda, MD, USA).

### Ultrasound Echocardiography

M- and B-mode para-sternal echocardiography of the left ventricle was performed according to established protocols at the Baylor College of Medicine Mouse Phenotyping Core using the MS550S transducer operating at 40 MHz on a VisualSonics Vevo 2100 system, and analyzed using Vevolab 5.7 software (Fujifilm VisualSonics).

### Histology and Immunofluorescence

Freshly dissected hearts were imaged for GFP fluorescence using a Zeiss LSM 780 confocal microscope. For fixation, hearts were retrogradely perfused with cardioplegic 20 mM KCl-PBS before perfusion with 10% neutral buffered formalin, followed by embedding in paraffin. Transverse sections (7 microns) were cut and mounted onto charged polylysine slides. A portion was stained with Masson's trichrome stain or H&E staining. Immunohistochemistry was performed by first deparaffinizing and rehydrating sections, followed by antigen retrieval and permeabilization in 0.5% Triton-X in PBS. Sections were blocked (10% donkey serum in phosphate-buffered saline [PBS], 0.1% Triton-X) and then incubated with primary antibody overnight at 4° C., and with secondary antibodies for 1 hour at room temperature before imaging. (FIG. 2A: anti-PCNA-Alexa-488, Santa Cruz Biotechnology Cat #sc-56; Mouse anti-cTnT-Alexa-647 conjugate, BD Pharmingen cat #565744; FIG. 2B: Rat anti-pHH3, Abcam Cat #ab10543; anti-rat Alexa 488, Thermo Fisher Scientific Cat #A-21208; FIG. 2C: Rabbit anti-Aurora B, Abcam Cat #ab2254; anti-Rabbit Alexa 488, Thermo Fisher Scientific Cat #A-21206.) Nuclei were stained with DAPI (4′,6-diamidino-2-phenylindole) (Thermo Fisher Scientific Cat #62248). Rhodamine-conjugated WGA was from Vector labs Cat #RL-1022. All imaging was performed with a Zeiss LSM 780 confocal microscope in the Optical Imaging and Vital Microscopy Core at Baylor College of Medicine (Houston, TX, USA).

For frozen sections (FIG. 1B, 1E), hearts were dehydrated with the 15% and the following 30% sucrose-PBS solution, and then placed into Tissue-Tek optical cutting temperature (OCT) compound (V.W.R. Cat #25608-930) before freezing over dry ice. Sections (10 microns) were cut and mounted on glass slides. For immunofluorescence staining, sections were fixed and permeabilized and then incubated with primary and secondary antibodies before imaging. (FIG. 1E: Rabbit DYKDDDDK Tag Antibody, Cell signaling technology Cat #14793, Mouse anti-cTnT-Alexa-647 conjugate, BD Pharmingen cat #565744.) Rhodamine-conjugated WGA was from Vector labs Cat #RL-1022. Nuclei were stained with DAPI. All imaging was performed with a Zeiss LSM 780 confocal microscope in the Optical Imaging and Vital Microscopy Core at Baylor College of Medicine.

### Nuclear Isolation for Sequencing

Nuclear isolation was performed as previously described. Briefly, fresh cardiac tissue was harvested on ice and was immediately cut into tiny pieces before performing Dounce homogenization in NP40 lysis buffer (10 mM Tris-HCl pH 7.4, 10 mM NaCl, 3 mM MgCl2, 0.1% NP-40, 1 mM DTT and RNase inhibitors). Homogenized solution was filtered and homogenate was mixed 1:1 with a 50 iodoxinal (5 volumes Optiprep [Sigma-Aldrich, Cat #D1556] with 1 volume Diluent [20 mM MgCl2; 60 mM Tris-Cl pH 7.4; 50 mM NaCl; 6% BSA; 6 mM DTT and RNase inhibitors]). Nuclei were isolated via density gradient centrifugation with Optiprep density gradient medium. After centrifugation for 12 min. at 10,000G, all nuclei isolated from a 30% to 40% interface were precleared with Protein-G Dynabeads (Thermo Fisher Scientific, Cat #10003D). Next, nuclei were immunoprecipitated with an anti-PCM1 (Sigma-Aldrich, Cat #HPA023370) antibody and Protein-G Dynabeads (washing 2 times with Wash Buffer [10 mM Tris-HCl pH 7.4; 10 mM NaCl; 3 mM MgCl2; 1% BSA; 0.1% Tween-20; 1 mM DTT and RNase inhibitors]) to enrich for CM nuclei as described previously.

### RNA Sequencing

RNA from bead-bound PCM-1(+) nuclei was collected using the RNEasy Plus Micro kit (Qiagen, Hilden, Germany). Nuclear RNA sequencing (RNA-seq) libraries were constructed using the Stranded RNA-seq Kit with Ribo-Erase (Kapa Biosystems Inc.) with custom Y-shaped adapters. Paired-end 2×75 bp sequencing was performed for RNA-seq libraries with an Illumina Nextseq instrument (DNA Link). Reads were first mapped to the mouse genome (mm10) using STAR (Dobin et al., 2013). Differential expression analysis was then carried out with DESeq2 (Love et al., 2014). Gene ontology analysis was performed using Metascape (Tripathi et al., 2015), and displayed using GOplot (Walter et al., 2015). Gene set enrichment analysis using publicly available data (Uosaki et al., 2015) was performed by interrogating the top 200 most enriched transcripts in either adult hearts relative to embryonic (E12-14); or embryonic relative to adult against our RNA-seq dataset (enrichment score is relative to control) (Mootha et al., 2003; Subramanian et al., 2005; Uosaki et al., 2015).

### In Vivo Drug Treatment

For in vivo treatment experiments, the ROCK inhibitor Y-27632 (StemCell Technologies, Inc. 72304) was diluted in mineral oil (Sigma, Lot #MKCH0156) and administered a dosage of 10 ug/g via subcutaneous injection into P9 mice. Mouse hearts were collected 2 days after drug treatment and were analyzed via pHH3 immunofluorescence, followed by quantification.

### Scar Quantification

Scar quantification was performed as previously described. Briefly, quantification was based on Masson's trichrome staining performed on representative serial cardiac sections spanning the entire heart. In each section, the percentage of scar (blue) was denoted by the angle of fibrotic tissue (in degrees, out of) 360°, measured using a protractor (using the middle of the heart as the center). The percentage of scarring was then averaged between all sections of a heart spanning from the beginning of the ventricles downward to the apex.

### Quantification and Statistical Analysis

Statistical tests, error bars, P-values, and n numbers are reported in the corresponding figure legends. Sample sizes were not pre-determined but were chosen based on previous publications. Mice were pre-determined to be excluded only if they had obvious anatomical or health abnormalities prior to experimental manipulation. To address randomness, any available (mutant or control) mice were included in the study. Control mice were AAV9-GFP-infected mice, AAV9-rtTA-infected mice, or mice without AAV9 virus infection, as indicated in the figure legends. Controls were littermates with or age-matched to experimental mice. No differences in variances were detected between any group in the reported experiments. One-way, two-tailed analysis of variance tests (ANOVA), followed by post-hoc tests were computed in Origin Pro (OriginLab Corporation). Fisher's exact test, Chi-squared test, and Mantel-Cox test were performed in Prism 5 (GraphPad). All graphs were generated in R or Microsoft Excel and presented using Prism. Cartoons were created in Biorender.

### REFERENCES

[1] Galvan, F. R., V. Barranco, J. C. Galvan, S. Batlle, Sebastian FeliuFajardo, and García, “epidemiology of myocardial infarction,” Intech, vol. i, no. tourism, p. 13, 2016.
[2] K. Thygesen, J. S. Alpert, and H. D. White, “Universal definition of myocardial infarction,” Circulation, vol. 116, no. 22, pp. 2634-2653, 2007.
[3] K. D. Poss, L. G. Wilson, and M. T. Keating, “Heart regeneration in zebrafish,” Science (80-.)., vol. 298, no. 5601, pp. 2188-2190, 2002.
[4] A. Abbate, R. Bussani, M. S. Amin, G. W. Vetrovec, and A. Baldi, “Acute myocardial infarction and heart failure: Role of apoptosis,” Int. J. Biochem. Cell Biol., vol. 38, no. 11, pp. 1834-1840, 2006.
[5] J. H. Van Berlo and J. D. Molkentin, “An emerging consensus on cardiac regeneration,” Nat. Med., vol. 20, no. 12, pp. 1386-1393, 2014.
[6] M. Dobaczewski, C. Gonzalez-Quesada, and N. G. Frangogiannis, “The extracellular matrix as a modulator of the inflammatory and reparative response following myocardial infarction,” J. Mol. Cell. Cardiol., vol. 48, no. 3, pp. 504-511, 2010.
[7] E. R. Porrello et al., “Transient regenerative potential of the neonatal mouse heart,” Science (80-.)., vol. 331, no. 6020, pp. 1078-1080, 2011.
[8] F. X. Yu and K. L. Guan, “The Hippo pathway: Regulators and regulations,” Genes Dev., vol. 27, no. 4, pp. 355-371, 2013.
[9] Z. Meng, T. Moroishi, and K. L. Guan, “Mechanisms of Hippo pathway regulation,” Genes Dev., vol. 30, no. 1, pp. 1-17, 2016.
[10] J. Wang, S. Liu, T. Heallen, and J. F. Martin, “The Hippo pathway in the heart: pivotal roles in development, disease, and regeneration,” Nat. Rev. Cardiol., vol. 15, no. 11, pp. 672-684, 2018.
[11] T. Heallen et al., “Hippo pathway deficiency reverses systolic heart failure after infarction,” 2017.
[12] Z. Lin et al., “Cardiac-specific YAP activation improves cardiac function and survival in an experimental murine MI model,” Circ. Res., vol. 115, no. 3, pp. 354-363, 2014.
[13] M. Xin et al., “Hippo pathway effector Yap promotes cardiac regeneration,” Proc. Natl. Acad. Sci. U.S.A, vol. 110, no. 34, pp. 13839-13844, 2013.
[14] A. Von Gise et al., “YAP1, the nuclear target of Hippo signaling, stimulates heart growth through cardiomyocyte proliferation but not hypertrophy,” Proc. Natl. Acad. Sci. U.S.A., vol. 109, no. 7, pp. 2394-2399, 2012.
[15] T. O. Monroe et al., “YAP Partially Reprograms Chromatin Accessibility to Directly Induce Adult Cardiogenesis In Vivo,” Dev. Cell, vol. 48, no. 6, pp. 765-779.e7, 2019.
[16] Z. Li et al., “Structural insights into the YAP and TEAD complex,” Genes Dev., vol. 24, no. 3, pp. 235-240, 2010.
[17] R. Forough, C. Scarcello, and M. Perkins, “Cardiac biomarkers: A focus on cardiac regeneration,” J. Tehran Univ. Hear. Cent., vol. 6, no. 4, pp. 179-186, 2011.
[18] P. Kurki, M. Vanderlaan, F. Dolbeare, J. Gray, and E. M. Tan, “Expression of proliferating cell nuclear antigen (PCNA)/cyclin during the cell cycle,” Exp. Cell Res., vol. 166, no. 1, pp. 209-219, 1986.
[19] A. Sawicka and C. Seiser, “Histone H3 phosphorylation-A versatile chromatin modification for different occasions,” Biochimie, vol. 94, no. 11, pp. 2193-2201, 2012.
[20] J. Livet et al., “Transgenic strategies for combinatorial expression of fluorescent proteins in the nervous system,” Nature, vol. 450, no. 7166, pp. 56-62, 2007.
[21] A. Loumaye, P. Lause, X. Zhong, T. A. Zimmers, L. B. Bindels, and J. Thissen, “Activin A Causes Muscle Atrophy through MEF2C-Dependent Impaired Myogenesis,” 2022.
[22] R. K. P. Natl, A. Sci, M. Light, and C. Isoform, “Characterization and Differential Expression of Human Vascular Smooth Muscle,” vol. 2, no. 1981, pp. 4027-4035, 1989.
[23] T. Aksel et al., “Ensemble Force Changes that Result from Human Cardiac Myosin Mutations and a Small-Molecule Article Ensemble Force Changes that Result from Human Cardiac Myosin Mutations and a Small-Molecule Effector,” pp. 910-920, 2015.
[24] D. Kwon et al., “SRF is a nonhistone methylation target of KDM2B and SET7 in the regulation of skeletal muscle differentiation,” Exp. Mol. Med., pp. 250-263, 2021.
[25] Y. Yatsuka et al., “A homozygous variant in NDUFA8 is associated with developmental delay, microcephaly, and epilepsy due to mitochondrial complex I deficiency,” Clin. Genet., vol. 98, no. 2, pp. 155-165, 2020.
[26] X. Song et al., “Article PDK4 dictates metabolic resistance to ferroptosis by suppressing pyruvate oxidation and fatty acid synthesis II II PDK4 dictates metabolic resistance to ferroptosis by suppressing pyruvate oxidation and fatty acid synthesis,” CellReports, vol. 34, no. 8, p. 108767, 2021.
[27] V. Chitu and E. R. Stanley, “Colony-stimulating factor-1 in immunity and inflammation,” vol. 1, no. II, pp. 39-48, 2006.
[28] T. B. Lear et al., “degradation KIAA0317 regulates pulmonary inflammation through SOCS2 degradation,” vol. 4, no. 19, 2019.
[29] G. S. Park et al., “An evolutionarily conserved negative feedback mechanism in the Hippo pathway reflects functional difference between LATS1 and LATS2,” Oncotarget, vol. 7, no. 17, pp. 24063-24075, 2016.
[30] B. Zhao et al., “Angiomotin is a novel Hippo pathway component that inhibits YAP oncoprotein,” Genes Dev., vol. 25, no. 1, pp. 51-63, 2011.
[31] T. Matsuda et al., “NF2 Activates Hippo Signaling and Promotes Ischemia/Reperfusion Injury in the Heart,” Circ. Res., vol. 119, no. 5, pp. 596-606, 2016.
[32] X. Yu and J. Chen, “DNA Damage-Induced Cell Cycle Checkpoint Control Requires CtIP, a Phosphorylation-Dependent Binding Partner of BRCA1 C-Terminal Domains,” Mol. Cell. Biol., vol. 24, no. 21, pp. 9478-9486, 2004.
[33] J. Li, Y. Li, F. Xu, B. Sun, L. Yang, and H. Wang, “Deubiquitinating enzyme PSMD14 facilitates gastric carcinogenesis through stabilizing PTBP1,” Exp. Cell Res., vol. 415, no. 2, 2022.
[34] Y. Ma et al., “LACTB suppresses melanoma progression by attenuating PPIA and YAP interaction,” Cancer Lett., vol. 506, no. 639, pp. 67-82, 2021.
[35] M. K. Tran, K. Kurakula, D. S. Koenis, and C. J. M. de Vries, “Protein-protein interactions of the LIM-only protein FHL2 and functional implication of the interactions relevant in cardiovascular disease,” Biochim. Biophys. Acta—Mol. Cell Res., vol. 1863, no. 2, pp. 219-228, 2016.
[36] B. E. Christian and L. L. Spremulli, “Mechanism of protein biosynthesis in mammalian mitochondria,” Biochim. Biophys. Acta—Gene Regul. Mech., vol. 1819, no. 9-10, pp. 1035-1054, 2012.
[37] K. Takahashi et al., “Heterogenous nuclear ribonucleoprotein H1 promotes colorectal cancer progression through the stabilization of mRNA of sphingosine-1-phosphate lyase 1,” Int. J. Mol. Sci., vol. 21, no. 12, pp. 1-17, 2020.
[38] Y. Cho, T. Ideue, M. Nagayama, N. Araki, and T. Tani, “RBMX is a component of the centromere noncoding RNP complex involved in cohesion regulation,” Genes to Cells, vol. 23, no. 3, pp. 172-184, 2018.
[39] G. Palacios et al., “Novel ALK fusion in anaplastic large cell lymphoma involving EEF1G, a subunit of the eukaryotic elongation factor-1 complex,” Leukemia, vol. 31, no. 3, pp. 743-747, 2017.
[40] X. Jing et al., “Cell-cycle-dependent phosphorylation of PrPS1 fuels nucleotide synthesis and promotes tumorigenesis,” Cancer Res., vol. 79, no. 18, pp. 4650-4664, 2019.
[41] S. Deng et al., “Overexpression of COX7A2 is associated with a good prognosis in patients with glioma,” J. Neurooncol., vol. 136, no. 1, pp. 41-50, 2018.
[42] T. Taira, Y. Saito, T. Niki, S. M. M. Iguchi-Ariga, K. Takahashi, and H. Ariga, “DJ-1 has a role in antioxidative stress to prevent cell death,” EMBO Rep., vol. 5, no. 2, pp. 213-218, 2004.
[43] E. Assémat, E. Crost, M. Ponserre, J. Wijnholds, A. Le Bivic, and D. Massey-Harroche, “The multi-PDZ domain protein-1 (MUPP-1) expression regulates cellular levels of the PALS-1/PATJ polarity complex,” Exp. Cell Res., vol. 319, no. 17, pp. 2514-2525, 2013.
[44] A. Kantardzhieva, S. Alexeeva, I. Versteeg, and J. Wijnholds, “MPP3 is recruited to the MPP5 protein scaffold at the retinal outer limiting membrane,” FEBS J., vol. 273, no. 6, pp. 1152-1165, 2006.
[45] D. Yu et al., “Myristoylated alanine-rich protein kinase substrate (MARCKS) regulates small GTPase rac1 and cdc42 activity and is a critical mediator of vascular smooth muscle cell migration in intimal hyperplasia formation,” J. Am. Heart Assoc., vol. 4, no. 10, pp. 1-18, 2015.
[46] J. Hülsken, W. Birchmeier, and J. Behrens, “E-cadherin and APC compete for the interaction with β-catenin and the cytoskeleton,” J. Cell Biol., vol. 127, no. 6 II, pp. 2061-2069, 1994.
[47] H. Hirata, H. Tatsumi, and M. Sokabe, “Mechanical forces facilitate actin polymerization at focal adhesions in a zyxin-dependent manner,” J. Cell Sci., vol. 121, no. 17, pp. 2795-2804, 2008.
[48] C. Ullmer, K. Schmuck, A. Figge, and H. Lübbert, “Cloning and characterization of MUPP1, a novel PDZ domain protein,” FEBS Lett., vol. 424, no. 1-2, pp. 63-68, 1998.
[49] M. Ernkvist et al., “The Amot/Patj/Syx signaling complex spatially controls RhoA GTPase activity in migrating endothelial cells,” Blood, vol. 113, no. 1, pp. 244-253, 2009.
[50] M. A. Estévez et al., “The neuronal RhoA GEF, Tech, interacts with the synaptic multi-PDZ-domain-containing protein, MUPP1,” J. Neurochem., vol. 106, no. 3, pp. 1287-1297, 2008.
[51] N. Mosaddeghzadeh and M. R. Ahmadian, “The rho family gtpases: Mechanisms of regulation and signaling,” Cells, vol. 10, no. 7, 2021.
[52] A. Piekny, M. Werner, and M. Glotzer, “Cytokinesis: Welcome to the Rho zone,” Trends Cell Biol., vol. 15, no. 12, pp. 651-658, 2005.
[53] O. Kanisicak et al., “Genetic lineage tracing defines myofibroblast origin and function in the injured heart,” Nat. Commun., vol. 7, 2016.
[54] T. Heallen et al., “Hippo signaling impedes adult heart regeneration,” Dev., vol. 140, no. 23, pp. 4683-4690, 2013.
[55] R. Gilsbach et al., “Dynamic DNA methylation orchestrates cardiomyocyte development, maturation and disease,” Nat. Commun., vol. 5, no. May 2014.
[56] A. Aharonov et al., “ERBB2 drives YAP activation and EMT-like processes during cardiac regeneration,” Nat. Cell Biol., vol. 22, no. 11, pp. 1346-1356, 2020.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A Yes-associated protein (YAP) variant comprising SEQ ID NO: 3.

2. A nucleic acid encoding the YAP variant of claim 1.

3. A vector comprising the nucleic acid of claim 2.

4. The vector of claim 3, wherein the nucleic acid is operably linked to a cell-specific or tissue-specific promoter.

5. The vector of claim 4, wherein the tissue-specific promoter is a cardiomyocyte-specific promoter.

6. The vector of claim 3, which is a viral vector.

7. The vector of claim 6, wherein the vector is an adeno-associated viral (AAV) vector.

8. A composition comprising (i) the vector of claim 3 and (ii) a carrier.

9. The composition of claim 8, which is a pharmaceutical composition.

10. The composition of claim 9, for use in a method of regenerating cardiomyocytes in an individual in need thereof.

11. The composition of claim 9, for use in a method of treating myocardial infarction in an individual.

12. A method of regenerating cardiomyocytes in a subject in need thereof, the method comprising delivering to cardiac tissue of the subject the composition of claim 9.

13. A method of treating myocardial infarction (MI) in a subject, the method comprising delivering to cardiac tissue of the subject the composition of claim 9.

14. The method of claim 12 or claim 13, wherein the composition comprises the vector of claim 5.

15. The method of claim 14, wherein the vector is an adeno-associated viral vector.

16. The method of claim 12, wherein the subject has arrhythmia, cardiomyopathy, heart failure, myocardial fibrosis, or myocarditis.

17. The method of claim 12, wherein the subject has experienced MI.

18. The method of any one of claims 12 to 17, wherein the composition is administered to the subject more than once.

19. A method of regenerating cardiomyocytes in a subject in need thereof, the method comprising delivering to cardiac tissue of the subject an AAV vector comprising the nucleic acid encoding the YAP variant of claim 1, operably linked to a cardiomyocyte-specific promoter.

20. A method of treating MI in a subject, the method comprising delivering to cardiac tissue of the subject an AAV vector comprising the nucleic acid encoding the YAP variant of claim 1, operably linked to a cardiomyocyte-specific promoter.


SEQUENCE LISTING (5 sequences)

SEQ ID NO: 1 - protein, 488 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 2 - protein, 488 aa, Synthetic Construct
MDPGQQPPPQPAPQGQGQPPSQPPQGQGPPSGPGQPAPAATQAAPQAPPAGHQIVHVRGDAETDLEALFNAVMNPKTANVPQTVPMRLRKLPDSFFKPPEPKSHSRQAATDAGTAGALTPQHVRAHAAPAALQLGAVSPGTLTPTGVVSGPAATPTAQHLRQAAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 3 - protein, 488 aa, Synthetic Construct
MDPGQQPPPQPAPQGQGQPPSQPPQGQGPPSGPGQPAPAATQAAPQAPPAGHQIVHVRGDAETDLEALFNAVMNPKTANVPQTVPMRLRKLPDAFFKPPEPKSHSRQAATDAGTAGALTPQHVRAHAAPAALQLGAVSPGTLTPTGVVSGPAATPTAQHLRQAAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 4 - protein, 487 aa, Synthetic Construct
DPGQQPPPQPAPQGQGQPPSQPPQGQGPPSGPGQPAPAATQAAPQAPPAGHQIVHVRGDAETDLEALFNAVMNPKTANVPQTVPMRLRKLPDSFFKPPEPKSHSRQAATDAGTAGALTPQHVRAHAAPAALQLGAVSPGTLTPTGVVSGPAATPTAQHLRQAAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 5 - protein, 487 aa, Synthetic Construct
DPGQQPPPQPAPQGQGQPPSQPPQGQGPPSGPGQPAPAATQAAPQAPPAGHQIVHVRGDAETDLEALFNAVMNPKTANVPQTVPMRLRKLPDAFFKPPEPKSHSRQAATDAGTAGALTPQHVRAHAAPAALQLGAVSPGTLTPTGVVSGPAATPTAQHLRQAAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL